(19) United States
(12) Patent Application Publication
(10) Pub. No.: US 2010/0111859 A1
(43) Pub. Date: May 6, 2010

(54) Visualization of Biological Material by the Use of Coated Contrast Agents

(76) Inventors: Oskar Axelsson, Hagan (NO); Kajsa Uvdal, Linkoping (SE)

Correspondence Address: PORTER WRIGHT MORRIS & ARTHUR, LLP;INTELLECTUAL PROPERTY GROUP, 41 SOUTH HIGH STREET, 28TH FLOOR, COLUMBUS, OH, 43215, US

(21) Appl. No.: 12/525,389

(22) PCT Filed: Jan. 10, 2008
(86) PCT No.: PCT/IB2008/050084

§ 371 Date: Nov. 30, 2009

Related U.S. Application Data
(60) Provisional application No. 60/899,995, filed on Feb. 7, 2007.

(51) Int. Cl. A61K 51/00; A61K 49/06; B05D 3/00; A61K 49/04; A61K 49/00

(52) U.S. Cl. 424/1.65; 424/9.32; 427/2.12; 424/9.42; 424/9.6; 977/773; 977/930

(57) ABSTRACT

A method for visualizing biological material, preferably by MRI, comprising the steps of: (i) bringing a population of coated nanoparticles into contact with said biological material, each of which nanoparticles comprises a) a metal oxide of a transition metal, said metal oxide preferably being paramagnetic and preferably comprising a lanthanide (+III) such as gadolinium (+III), and b) a coating covering the surface of the core particle, and (ii) recording the image; wherein the coating is hydrophilic and comprises a silane layer which is located next to the surface of the core particle and comprises one or more different silane groups which each comprises an organic group R and a silane-siloxane linkage where a) R comprises a hydrophilic organic group R′ and a hydrophobic spacer B, b) O is oxygen directly binding to a surface metal ion of the metal oxide, and c) C is carbon and is also part of B. A composition for visualization and methods for the manufacture of the nanoparticles and core particles are also disclosed. Visualization includes imaging by MR, CT, X-ray, near IR fluorescence, PET, microscopying etc with the largest advantages accomplished for in-vivo imaging.

### TECHNICAL FIELD

The invention relates to nanoparticles that are to be used as contrast agents for visualizing or imaging biological material. The nanoparticles are typically paramagnetic with each nanoparticle construed of a core particle and a coating covering the surface of the core particle. Individual core particles have surfaces that expose a metal oxide comprising a transition metal ion. The metal oxide is typically paramagnetic and the transition metal ion is preferably a lanthanide (+III), such as gadolinium (+III). Main aspects of the invention are a) methods for the visualization of biological material utilizing the nanoparticles, b) compositions of the nanoparticles, c) methods for the manufacture of the nanoparticles (coated core particles) and/or of the core particles to be coated, d) use of the nanoparticles for the manufacture of a composition intended for in vivo visualization of biological material etc. The invention is in particular beneficial for magnetic resonance imaging (MRI) and other imaging techniques such as X-ray, computer tomography (CT) etc.

The term “transition metal” will be used in a broad sense in the context of the invention and thus includes elements between group 2b and 3a of the periodic system, i.e. groups 3b, 4b, 5b, 6b, 7b, 8, 1b and 2b with the lanthanides and actinides being part of group 3b.

### TECHNICAL BACKGROUND

The principle of Magnetic Resonance Imaging of biological material, MRI, is the detection of the nuclear magnetization of the hydrogen nuclei of water molecules that are present in the material. The main advantage of MRI over X-ray imaging is the enhanced contrast between different soft tissues. This contrast has at least three different origins. The trivial is the proton density but, more interestingly, the recovery times (relaxation times) T of the magnetization, T₁ (along the main magnetic field) and T₂ (perpendicular to the main magnetic field) are important contributors to contrast. Both T₁ and T₂ are sensitive to the viscosity, magnetic susceptibility, temperature of the material and presence of other magnetic entities.

A decrease in T₁ and T₂ leads to an increase and decrease, respectively, in the measured MR signal. If a spin echo sequence is used for the measurement, the signal S expressed as a function of scanning parameters can in simplified form be expressed as:

S(TR,TE)=ρe^{-TE/T2}(1−e^{-TR/T1})

where ρ=spin density, TE=echo time and TR=repetition time. Paramagnetic contrast agents are used to shorten relaxation times to allow more signal to be collected in a given period of time. This enhanced signal can be utilized to improve the resolution in the images or to use a shorter acquisition time. MRI contrast agents have effects on both T₁ and T₂ but some agents are selective in the sense that their effect on T₁ is stronger than on T₂ or vice versa. Paramagnetic metal ions, such as the gadolinium ion (Gd³⁺) in the form of chelates and also particles of insoluble salts of certain metals, such as gadolinium oxide (Gd₂O₃) and iron oxide (Fe₂O₃), have been suggested as contrast agents in MRI. Gadolinium (III+) with a predominant effect on T₁ has been used as a positive contrast agent (increased MR signal) and the oxide form of Iron (III+) with a predominant effect on T₂ as a negative contrast agent (decreased MR signal). The relaxation rate (1/Tᵢ, i=1, 2 for hydrogen) is proportional to the concentration C of the used contrast agent, i.e.

1/Tᵢ (observed)=1/Tᵢ (inherent)+rᵢC

where 1/Tᵢ (observed) is the relaxation rate in the presence of the contrast agent, 1/Tᵢ is the inherent tissue relaxation rate, and rᵢ is a proportionality constant called the relaxivity of the contrast agent. See Engström et al. (Magn Reson Mater Phy 19 (2006) 180-186 and WO 2006031190 (Uvdal et al.) and references cited therein.

The effect of a particular contrast agent on the relaxation times of the hydrogen nuclei in a sample and on the MR image depends in a complex way on a number of factors, such as the magnetic moment of the relaxation agent, the electron relaxation time, the ability to co-ordinate water in the inner and/or outer coordination sphere, rotational dynamics of the paramagnetic agent, diffusion and water exchange rate. For well behaved systems, this is described quantitatively by the Solomon-Bloembergen-Morgan theory (The Chemistry of Contrast Agents in Medical Magnetic Resonance Imaging, Wiley 2001, Eds. Andre E. Merbach, Eva Toth).

Contrast agents that are used clinically in vivo are typically administered to a patient by injection with preference for intravenously. This means that the metal part of the contrast agent has to be given in a form that is not harmful for the patient and remain for a sufficiently long period of time in the patient for the intended use to be performed. The agent also has to be capable of being transported in vivo to the desired part of the patient.

To lower the toxic effects of free gadolinium ions, Gd³⁺ has been used clinically in stably chelated form, typically as a diethylene triamine penta acetic acid chelate (DTPA) or as chelates with congeners of DTPA, such as tetraazacyclododecane tetra acetic acid, DOTA and other analogues of these chelators. Nanoparticles containing Gd₂O₃ have so far not been approved for clinical use. The less toxic iron (III+) has been used clinically in the form of Fe₂O₃ nanoparticles. Larger Fe₂O₃ nanoparticles are quickly accumulated in the reticuloendothelial system (RES), and hence have short blood lifetimes and have found use in liver imaging. Smaller Fe₂O₃ nanoparticles have longer blood lifetimes since they are escaping the RES, and have been considered to have a broader potential for imaging in vivo. With respect to clinical use of nanoparticulate forms of Fe₂O₃, the particles have been coated in order to increase their stability against agglomeration and to make them invisible to the immune system. The coatings have typically been biodegradable since this would facilitate degradation of the metal oxide core and thus also facilitating the removal of the particles and the metal ions from a patient's body. However, this is not applicable to particles containing non-endogenous metal ions that often are highly toxic. In such cases it seems more reasonable to rely on renal excretion for the safe removal of the particles from the body. Renal excretion requires the particles to be very small. Depending on the method of measurement, surface charge and unknown material factors, the typical cut-off size for renal filtration is about 6-8 nm in diameter (O'Callaghan, C. Brenner, B. M., “The Kidney at a Glance, Blackwell Science, 2000 p 13) although larger particles, e.g. up to 10 nm may be excreted due to plasticity effects.

Due to problems with the current generation of contrast agents an alternative strategy for encapsulation of metal ions in contrast agents of the type discussed above would be desirable. Stably coated metal oxide nanoparticles, may be the solution to this problem. (Marckmann P. et al., J Am Soc Nephrol. 17(9):2359-62. September 2006. Epub August 2006).

For coated MRI nanoparticles it is imperative that the coating doesn't prevent magnetic dipolar coupling between the metal oxide core with water molecules in the surrounding medium or the relaxivity of the particles will be low. For nanoparticles that are intended to be renally excreted it will be a challenge to design a coating that has both a sufficient stability for this kind of excretion and provides ample opportunity for association of water molecules to give improvements over current MRI contrast agents.

### BACKGROUND PUBLICATIONS OF INTEREST

1. WO 2005088314 (Perriat et al).
2. WO 2006031190 (Uvdal et al).
3. US 2004156784 (Haase).
4. U.S. Pat. No. 4,770,183 (Groman).
5. US 20030180780 (Feng et al).
6. U.S. Pat. No. 6,638,494 (Pilgrimm).
7. CN 1378083 (Sun et al; Chemical Abstracts 8 Mar. 2004, XP002300295 extract from STN data base accession no 2004:184856).
8. Bazzi et al., J. Colloid Interface Sci. 273 (2004) 191-197.
9. Bridot et al., J. Am. Chem. Soc. 2007, 129, 5076-5085. Publ Mar. 31, 2007.)
Feng et al., Anal. Chem. Am. Chem. Soc, Columbus US, 75(19) (2003) 5282-5286.
11. Jun et al., Nanomaterials for Cancer Diagnosis, Ed. Challa S. S. R. Kumar, Nanotechnologies for the Life Science, Vol 7 (147-173) 2007. Wiley-VCH Verlag GmbH & Co. KGaA, Weinheim, Germany.
12. Louis et al., Chem. Mater., 17 (2005) 1673-1682.

All patents, patent applications and publications cited in this specification are incorporated by reference in their entirety.

### SUMMARY OF THE INVENTION

A main aspect of the invention is a method for visualizing biological material, preferably by MRI, as generally outlined in the introductory part. The method comprises the steps of:
(i) bringing a population of nanoparticles in contact with said biological material, each of which nanoparticles comprises a) a core comprising a surface in which a metal oxide is exposed, and b) a coating covering the surface of the core, and
(ii) recording the image, e.g. in a per se known manner.

The metal oxide comprises a transition metal ion and is preferably paramagnetic for instance with said transition metal ion being paramagnetic. The transition metal ion is preferably a lanthanide (+III), such as gadolinium (+III). A core particle in the population typically is homogeneous with respect to occurrence of the metal oxide, i.e. the metal oxide is in the ordinary variants located all throughout the body of a core particle and not only to its surface. The core particles and the nanoparticles may be super paramagnetic if the metal ions and the size of the particles are properly selected.

In other aspects, the invention is directed to a method of coating a population of core particles and to compositions for visualizing biological material. Other aspects of the invention will be more apparent in the Detailed Description.

### DETAILED DESCRIPTION

The invention is directed to methods and compositions for visualizing biological material, and to methods of coating a population of core particles.

There is a need for improved nanoparticles that can be used as contrast agents in imaging techniques of the kinds described herein with particular emphasis on MRI. This includes novel metal oxide nanoparticles that will facilitate an increased contrast in the images and an increased signal which can be translated to either shorter acquisition times, higher spatial resolution or a reduction in dose of the contrast agent. Spatial resolutions down to 1 mm voxel linear dimension (size) should thus easily be reached by the use of certain embodiments of the invention with scanning measurements typically being performed during time periods of up to two hours, such as up to 45 minutes. In certain embodiments, resolutions down to 0.1 mm voxel linear dimension and even lower, but realistically in most cases above 0.01 mm voxel linear dimension, are desirable. Adequate speed and signal to noise ratio are also advantageous to enable several applications in anatomical imaging. Particularly, coronary angiography with resolutions within the ranges given are of great clinical benefit.

It is also advantageous to use nanoparticulate contrast agents for enhanced contrast in tumour imaging and/or for imaging other tissues showing enhanced leakiness to large entities and/or being delineated by a less organized endothelium compared to normal tissue. In one embodiment, such agents are useful for monitoring the response to anti-angiogenic therapy (H. Daldrup-Link et al., Academic Radiology, Volume 10, Issue 11, Pages 1237-1246).

It is also advantangeous to lower toxicity which is strongly linked to enhanced stability against release of metal ions (non-degradable and stable coatings) and elimination of the nanoparticles from patients by renal filtration without in-vivo release of toxic metal ions. For the MRI application, it is advantageous to accomplish lowered toxicity and renal excretion of the particles while maintaining efficient magnetic dipolar coupling between metal ions of the nanoparticles and hydrogen nuclei in the surrounding liquid medium, i.e. two objectives that require effects that would neutralize each other if the nanoparticles are not properly designed.

Various embodiments of the invention provide one or more of these advantages.

A main aspect of the invention is a method for visualizing biological material, preferably by MRI, as generally outlined in the introductory part. The method comprises the steps of:
(iii) bringing a population of nanoparticles in contact with said biological material, each of which nanoparticles comprises a) a core comprising a surface in which a metal oxide is exposed, and b) a coating covering the surface of the core, and
(iv) recording the image, e.g. in a per se known manner.

The metal oxide comprises a transition metal ion and is preferably paramagnetic for instance with said transition metal ion being paramagnetic. The transition metal ion is preferably a lanthanide (+III), such as gadolinium (+III). A core particle in the population typically is homogeneous with respect to occurrence of the metal oxide, i.e. the metal oxide is in the ordinary variants located all throughout the body of a core particle and not only to its surface. The core particles and the nanoparticles may be super paramagnetic if the metal ions and the size of the particles are properly selected.

The nanoparticles and their coating and the core particles as such are also described in our U.S. provisional patent application Ser. No. 60/899,995 filed on Feb. 7, 2007 with the title “Compositions containing metal oxide particles and their uses”.

A main characteristic feature of the method is that the coating is hydrophilic and comprises next to the surface of the core particle a silane layer which contains one, two or more different silane groups. Each of these groups comprises an organic group R (i.e. R¹, R², R³ etc) bound to the surface of the core via a silane-siloxane linkage —O—Si—C—, where a) the oxygen atom O is directly binding to a surface metal ion of the core particle, and b) the carbon atom C, typically sp³-hybridised, is part of a hydrophobic spacer B and is directly binding to one or two other carbons. Each of the different organic groups R comprises the hydrophobic spacer B and a hydrophilic organic group R′ (i.e. ═R^{1′}, R^{2′}, R^{3′} etc) directly attached to B. The hydrophobic spacer B may differ for the different groups R and R′, i.e. B may be=B¹, B², B³ etc). The one or two carbons binding to the carbon atom C are preferably sp³-hybridised, and depending on the length of B, they are part of B and/or R′. The coating and/or only the silane layer next to the core surface preferably have the dimension of a monolayer (with respect to silane groups).

The coating may also exhibit hydrophobic silane groups.

The hydrophobic spacer B is a pure hydrocarbon spacer and should be relatively short, for instance complying with

CₙH₂ₙ₋₂ₐ— (Formula I)

where one, two or more of the hydrogens possibly is/are substituted with a lower alkyl or a lower alkylene group, respectively. The range for n is integers in the interval 1-10 with preference for 1, 2, 3, 4 or 5. The range for a is integers 0, 1, 2, 3 etc with a≦n. The term “pure” in this context means that B only contains carbon and hydrogen. The spacer B becomes —CₙH₂ₙ— for a equal to 0.

Lower alkyl, lower alkoxy, lower alkylene, and lower acyl (in particular alkanoyl) in the context of the invention will mean C₁₋₁₀ alkyl, C₁₋₁₀ alkoxy, C₁₋₁₀ alkylene or C₁₋₁₀ acyl groups. If not otherwise indicated these groups may be substituted with heteroatom-containing groups (heteroatom O, N, S) as discussed below for R₁ and R_{1′}.

The coating is created by reacting the core particles with one or more silane reagents. If a silane reagent has only one organic group R as in preferred variants of the invention, the R group will be stably attached to one or more metal ions in the core surface via three oxygens (R—SiO₃). The layer next to the surface and also the coating as such can be further stabilized by comprising polysiloxane, for instance introduced by reaction with a reticulating reactive silicate as described under the heading “Coating Procedure”. A preferred polysiloxane typically defines a cross-linked network (typically 3-D or 2-D) that effectively helps in stitching up any defects in the layer next to the surface thereby rendering release of metal ions from the core more difficult. For maximal stability, it is imperative that the layer next to the core surface and/or the coating as such is very dense, preferably similar to close packing.

The polysiloxane, with or without appending silane groups, may define an additional layer on top of the silane layer that is next to the core surface. A silane group that is present in this second layer is typically linked to the surface of the core particle via siloxane linkages placing two or more silicon atoms [—(Si—O—)ₙ, where n is an integer ≧2] between the organic part of the silane group and the surface of a core particle.

The number of surface metal ions can easily be derived for different crystal states and kinds of metal oxides. Provided the metal oxide is gadolinium oxide the number of surface gadolinium ions can be estimated according to:

$$N = \frac{4}{3 M} d π ( {r}^{3} - {( r - l )}^{3} ) {N}_{A}$$

where d is the density of gadolinium oxide (7.41 g/cm³=7.41×10⁶ g/m³), r is the radius of the core particle, 1 is the distance between the most prominent crystal planes (the (222) planes in this case, 3.12×10⁻¹⁰ m), M is the molecular weight for GdO_{1.5}=181 g/mol and N_{A} is Avogadro's number 6.022×10²³/mol. This formula is based on the assumption that the bulk density of gadolinium oxide is similar to the density of nanoparticulate material and that the particles are spherical so it is not to be taken as literal truth but as a reasonable estimate.

A Gd₂O₃ particle with 2 nm diameter will have 69 surface gadolinium ions and contain 1.5 oxide ions for every gadolinium ion. For a siloxane linkage carrying three oxygens (deriving from a silane reagent comprising one organic silane group and three alkoxy groups), it is reasonable to assume that that there is room for one siloxane linkage for every two surface gadolinium ions. Complete coverage of the surface of the particle would then require around 34 silicon atoms for a two nanometer core particle. A calculation analogous to the above gives that the total number of gadolinium ions in the 2 nm particle should be 103. The silicon to gadolinium molar ratio should then be expected to be 34/103=0.33 for a particle of this size with complete coverage (maximum Si:Gd molar ratio or complete coverage value for a 2 nm particle). Gadolinium oxide nanoparticles with a monolayer of silane should thus show a silicon to gadolinium ratio of ≧50% of the complete coverage value for the particle size concerned, with preference for higher percentages such as ≧80% and even higher such as ≧90%. The complete coverage value (=maximum Si:Gd molar ratio) are summarized in table 1 for gadolinium oxide particles with a range of core sizes.

TABLE 1

| Particle | No Gd | No Surface Gd | No Si | Maximum Si:Gd |
|---|---|---|---|---|
| diameter (nm) | ions total | ions | atoms | molar ratio |
| 2 | 103 | 69 | 34 | 0.330 |
| 3 | 348 | 175 | 87 | 0.250 |
| 4 | 826 | 329 | 164 | 0.200 |
| 5 | 1613 | 531 | 265 | 0.160 |
| 7 | 4427 | 1081 | 540 | 0.120 |
| 10 | 12908 | 2268 | 1143 | 0.088 |
| 15 | 43566 | 5214 | 2607 | 0.060 |
| 20 | 103268 | 9367 | 4683 | 0.045 |

If the coating comprises an additional layer comprising polysiloxane with or without organic silane groups, the actual silicon to gadolinium ratio defined above will be above the complete coverage value, i.e. exceed 100%, but will preferably be ≦1000%, such as ≦750% or ≦500% or ≦250% or ≦150%.

Analogous calculations can be performed also for populations of nanoparticles in which the core particles are based on other transition metal oxides, such as other lanthanide oxides. Essentially the same percentage intervals for the molar ratio between silicon and metal ion will apply as for the gadolinium oxide nanoparticles above. In the case wherein a particle contains two or more different metal ions, the calculations have to be based on the distance between the crystal planes that correspond to the most prominent crystal faces.

The above-mentioned ranges apply to populations of particles with the presumption that all particles have the mean geometric diameter as diameter.

In the coating the molar ratio between silicon and carbon bound directly to silicon (silane carbon) is ≧1 and typically ≦10, such as ≦5 or ≦2.5, with preference for ≦1.5 or ≦1.25 or ≦1.1, provided monoalkyl silane reagents possibly combined with other reticulating silicates have been used for the coating process. The ratio may be <1 if the coating process has comprised reaction with dialkyl- and/or trialkyl silane reagents. These ranges in particular apply to the silane monolayer that is present next to the core surface, i.e. the silicon atom is part of a —O—Si—C— or —O—Si—O— linkage in which at least one of the oxygens binds directly to a metal ion of the core surface and remaining oxygen(s) if any bind to another silicon atom.

The coating typically has a thickness which is ≦10 nm, such as ≦5 nm or ≦1 nm or ≦0.7 nm with a typical lower limit of 0.1 nm or 0.5 nm. The thickness of a monolayer depends on the size of R (and R′) and is typically ≦5 nm or ≦1 nm or ≦0.7 nm with a typical lower limit of 0.1 nm or 0.5 nm. Thickness in this context refers to the mean thickness of the coat of the nanoparticles of the population.

Nanoparticles of the population used in the method, i.e. the coated core particles, typically have a mean hydrodynamic diameter (size) within the interval ≦20 nm or ≦10 nm or ≦6 nm. The actual measured size of the nanoparticles will depend on the composition of the coating and the environment in which the nanoparticles are present, for instance the coating may have a propensity to swell in an aqueous medium (hydrophilic coatings). Particularly preferred coated variants comprise populations of nanoparticles that have mean hydrodynamic diameters (sizes) within the range of ≦7 nm, such as 3-6 nm in order to promote elimination of the nanoparticles by renal filtration when present in a patient. Though, one should be aware that a coated particle with a larger hydrodynamic diameter than 7 nm, for instance up to 8 nm or up to 10 nm, may also be filtered out due to deformations or, put another way, the effective filtration diameter is not necessarily the same as the hydrodynamic diameter.

The sizes of the coating and of the nanoparticles refer to measurements carried out in deionised water by dynamic light scattering (DLS).

The population of nanoparticles (coated core particles) used in the method are preferably monodisperse in the sense that ≧25%, such as ≧50% with preference for ≧75% or ≧90% or ≧95% of the nanoparticles have sizes within a size interval with the width of ≦10 nm with preference for ≦5 nm or ≦3 nm or ≦2 nm or ≦1 nm and/or a size distribution with ≧75% preferably with ≧90%, such as ≧95% of the nanoparticles within a size range that is ±75%, such as ±50% or ±25% or ±10% of the mean nanoparticle size. For most in vivo applications the preferred populations of nanoparticles will have a size distribution with ≦10%, preferably ≦5%, of the nanoparticles being ≦4 nm, such as ≦3, nm or ≦2 nm and/or ≧6 nm, such as ≧7 nm or ≧8 nm or ≧9 nm or ≧10 nm. Populations of nanoparticles that are not monodisperse are polydisperse.

### The Organic Part of the Coating

The hydrophilic coating of the invention typically exhibits a plurality of polar functional groups containing one or more heteroatoms selected among oxygen, nitrogen, sulphur, and phosphorous. These heteroatoms may be present in mono-, bi- and trivalent functional groups such as ether, thioether, hydroxyl, carbonyl e.g. carboxylic acid and salts, amides and esters thereof, carbamido, carbamate, keto etc, phosphonic acid and salts, esters and amides thereof, sulphonic acid and salts, esters and amides thereof, sulphone etc. The ratio (“hydrophilicity ratio”) between the number of the heteroatoms mentioned above and the number of carbon atoms in a hydrophilic coating is typically ≧0.2, such as ≧0.3, with the contribution from the hydrophobic spacer B not being included. Coating structures containing substructures in which there are one or more groups selected amongst amide, hydroxyl and/or repetitive ethyleneoxy groups either alone or in combination with each other are of particular value due to their polar nature which allow them to associate with a large number of water molecules which will have an advantageous effect on the relaxivity of the particles. For substructures containing two or more of these groups, for instance of the same kind or different kinds there should be a linkage of zero, one, two, three, or four atoms between heteroatoms (nitrogens and/or oxygens) of adjacent groups, Such a linkage typically comprises one, two or three carbon atoms. Preferred such substructures contains one, three, four or more amide groups and/or and one, three, four or more hydroxy groups.

The functional groups and the ranges for the hydrophilicity ratio mentioned above for the coating are inherently also applicable to the hydrophilic organic group R′ that is part of the organic group R. Thus the hydrophilic organic group R′ typically comprises a carbon chain which at one, two or more positions a) is interrupted by an at least bivalent functional group containing an heteroatom (O, N, S and P), and/or b) comprises a carbon that is (i) substituted with a hydroxyl or a lower alkoxy or a lower hydroxyalkoxy group, or amino or substituted amino, such as lower C₁₋₁₀ alkylamino (mono-, di- and trialkylamino), (ii) constitutes a branching point of the carbon chain and a branch group that comprises structural elements selected from the same structural elements as may be present in the hydrophilic organic group The hydrophilic organic group R′ may be straight, branched or cyclic. The lower alkyl and lower alkoxy groups may be substituted with heteroatom-containing functional groups, for instance as discussed for R₁ and R_{1′} below.

The hydrophilic organic group R′ is attached to the spacer B via a) a bivalent heteroatom-containing functional group, or b) an sp³-carbon atom directly binding to a heteroatom. Both of these linking groups are considered to be part of the hydrophilic group R′.

Typical at least bivalent functional heteroatom-containing groups are ether (—O—), thioether (—S—), and amido (—CO—NR₁—, —NR₁—CO—) where R₁ has the same meaning as given below, and at least bivalent forms of the functional groups given above and of the groups X given below.

In a hydrophilic group R′ each sp³-hybridised carbon typically binds at most one heteroatom (O, N or S).

The carbon chain discussed above in the hydrophilic group R′ typically has at most 35 atoms linked to each other in series (including carbons and interrupting heteroatoms).

The coating preferably exhibits charged groups giving the nanoparticles a net charge in order to prevent them from aggregating in solution. The number and kind of charges should be selected to give the population of the nanoparticles an absolute zeta potential ≧20 mV, such as ≧30 mV, in salt free water (deionised water). The charged groups may be selected from negatively and/or positively charged groups, with preference for the former. Examples of preferred negatively charged groups (anionic) are: carboxy/carboxylate (—COOH/COO⁻), phosphonate (—PO₃²⁻/—PO₃H⁻/—PO₃H₂), sulphonate (—SO₃⁻/—SO₃H) where the free valence binds to carbon with preference for sp³-hybridised carbon. Examples of positively charged groups (cationic) are various ammonium groups, such as primary, secondary, tertiary and quaternary ammonium group with preference for the quaternary ones because they are charged in the complete pH interval of interest for in vivo applications. The charged groups are preferably present in at least one of the one or more different hydrophilic organic R′-groups.

The mean value for the molar ratio (for a population of particles) between charged R′ groups and uncharged R′ groups is typically ≧0.05, such as ≧0.1 or ≧0.5, and ≦20, such as ≦10 or ≦2, preferably with respect to the ratio between negatively charged and uncharged R′ groups.

The hydrophilic group R′ in R is in preferred variants of the method selected amongst groups complying with the formula:

-(ACH₂CH₂)ₚ(OCH₂CH₂)ₘA′ₒ(CH₂)ₙX (Formula II)

where
a) n′ is an integer 0-15, preferably 1-5,
b) m is an integer 0-10, preferably 2-5,
c) o and p are equal or different integers 0 or 1, with the proviso that one of them preferably is 0 when m is 0;
d) A and A′ are heteroatom-containing bivalent functional groups as defined above with heteroatoms selected amongst oxygen, nitrogen and sulphur, with preference for ether, thioether and amino, and
e) X is selected amongst carboxylate alkylesters, phosphonate alkyl esters (mono and dialkyl), sulphonate alkylesters, N-alkyl amides (mono and dialkyl), N-alkyl phosphonic acid amides (mono- and dialkyl), N-alkyl sulphonamides (mono- and dialkyl), alkyl ethers and the corresponding hydrolysed forms.

The group X thus may be selected amongst —COOR₁, —PO(OR₁)(OR_{1′}), —SO₂(OR₁), —CO(NR₁R_{1′}), R₁CO(NR_{1′}—), —PO(NR₁R_{1′}), R₁PO(NR_{1′}—), —SO₂(NR₁R_{1′}), R₁SO₂(NR_{1′}—), and —OR₁, R₁ and R_{1′} are in various Xs independently selected amongst hydrogen and linear, branched or cyclic C₁₋₁₀ alkyl optionally carrying (=being substituted with) one or more hydroxyl and/or amino groups and/or containing a carbon chain that is interrupted at one or more positions by insertion of a heteroatom selected from oxygen, nitrogen or sulphur or some other at least bivalent heteroatom-containing functional group of the type given in this specification.

The hydrophilic group R′ may also contain one or more branchings that are obtained by replacing one or more of the hydrogens in formula II with a group complying with formula II.

Preferred hydrophilic organic groups R′ and combinations are:
a) —CH₂CH₂COOCH₃ and/or —CH₂CH₂COOCH₂CH₃, either alone or in combination with —CH₂CH₂COOH,
b) —CH₂CH₂PO(OCH₂CH₃)₂ and/or —CH₂CH₂PO(OCH₃)₂, either alone or in combination with —CH₂CH₂PO(OH)₂,
c) —CH₂CH₂(OCH₂CH₂)ₙOH (n=an integer 1-5) and/or —CH₂CH₂(OCH₂CH₂)_{n″}OCH₃ (n″=an integer 1-5), either alone or in combination with —CH₂CH₂COOH, and/or —CH₂CH₂PO(OH)₂ and/or —CH₂CH₂S O₃H.
d) —CH₂CH₂CH₂NHCONHR₁, where R₁ has the same meaning as above with preference for being —CH₂CH₂OH.

The aim with the coat of the present invention is to improve the stability of the core particles with respect to tendency to release metal ions. Therefore, in one embodiment, the nanoparticles of the present invention should have a reduced release of metal ions in aqueous media giving them at least the same life-time or a life-time that is at least 150%, such as at least 200% or at least 300% longer, than the life-time for the corresponding uncoated forms (bare forms, core forms). These comparisons are between results achieved under the same conditions as elaborated in the experimental part with life-time measured as the time it takes for reducing the concentration/amount of one or more of the transition metal ions of the metal oxide of the core particles when present in an aqueous suspension to 50% of the starting concentration/amount (half-life time, t_{1/2}).

The coating may or may not comprise a so-called targeting group for targeting a certain structure of a biological material and/or a so-called label group, e.g. a fluorescent or a luminescent group. The coatings of nanoparticles not being intended for targeting or for assay purposes involving detection of labels typically are devoid of polypeptide structure, nucleic acid structure, lipid structure, polysaccharide structure, and/or systems of conjugated double bonds such as in aromatic systems and α-β unsaturated carbonyl structures.

Nanoparticles that are to be used as contrast agents in the body of an animal or an organ thereof and administered via the blood circulation should be able to remain in the blood circulation for a time sufficient for the desired image to be recorded. The exact desired lifetime will depend on the part of the body/organ to be imaged and species, such as humans, mice, rats, rabbits, guinea pigs etc. As a general guideline, suitable lifetimes (t_{1/2}) of this kind are typically found in the interval of ≧5 minutes, such as ≧10 minutes, or ≧30 minutes or ≧1 hour or more with upper limits for lifetimes (t_{1/2}) typically being 2 hours, 24 hours, 48 hours, 62 hours or more, with particular emphasis of a clearance of ≧80%, such as ≧90% or ≧99% in 48 hours from the living body to which the nanoparticles have been administered.

### Compositions

The compositions of the population of nanoparticles described in this specification to be used for visualization constitute the second main aspect of the invention. In these compositions the population of nanoparticles are A) mixed with a buffer system, e.g. physiologically acceptable, and/or with a suitable non-buffering salt, e.g. physiologically acceptable, and/or a carbohydrate, such as mono- or polysaccharide (containing one, two, three or more monosaccharide units), and/or B) in dry powder form or as a dispersion in a liquid, e.g. aqueous liquid such as water. The powder form may have been obtained by lyophilization, air drying, spray-drying etc of a dispersion containing the particles and the proper liquid medium. The powder form of the inventive composition is typically dispersible in the liquid in which the particles are to be used. Such liquids are typically physiologically acceptable and/or aqueous (e.g. water). Examples of potential useful buffer systems to be included in liquid dispersion media or in compositions in dry form (e.g. powder form) are illustrated with 2-morpholino-ethanesulphonic acid (MES), 4-(2-hydroxyethyl)piperazine-1-ethane sulfonic acid (HEPES), and trishydroxymethylmethylamine (TRIS). Phosphate buffers may adversely affect the particles and if used might require more stable coatings than other buffers. Buffers that enhance aggregation and sedimentation should be avoided. Suitable carbohydrates are water-soluble, such as glucose, lactose, saccharose, trehalose, etc.

The composition may also comprise other ingredients, such as one or more populations of other particles, including other nanoparticles.

In dispersed variants of the innovative compositions, e.g. with the nanoparticles dispersed in a physiologically acceptable aqueous liquid phase, the optimal total concentration of the metal ion of the metal oxide present in the core particles could reach ≧10 mM with increasing preference for ≧50 mM or ≧100 mM or ≧500 mM or ≧1 M. Upper limits are 4 M or 10 M. Even higher concentrations can be envisaged. The composition to be used in the inventive method typically has a viscosity ≦50 mPas, such as ≦25 mPas or ≦15 mPas, at a concentration of 0.5 M of the metal ion of the nanoparticles, i.e if the composition is a liquid dispersion in which the concentration of the metal ion is above 0.5 M, a viscosity in this range is achievable upon dilution to 0.5 M. For manual bolus injection it is important with a viscosity of no more than 25 mPas, which is the practical limit. To achieve this, it is important that the coating is optimally thin for the particle preparation to be compatible with the demands for high concentration combined with low viscosity. For many contrast agents this limit is reached when the volume fraction of contrast agent particles/molecules in the injectable formulation/composition is around 30%. For a particle preparation with a 5 nm diameter Gd₂O₃ core (containing 1613 Gd ions according to table 1) and a 2 nm coating we get only about 5% volume fraction for a dispersion that is 1 M in metal ion of the nanoparticles. This is very advantageous over classical macromolecular contrast agents where gadolinium chelates are coupled to a macromolecule and the structures are much less compact than the nanoparticles of the current invention.

A further advantage of the inventive contrast agent is that the osmolality can be substantially lower than for particularly Magnevist (GdDTPA) which is as high as 1960 mOsm. With a particulate contrast agent the osmolality will no longer be very dependent on the total number of particles in solution but rather of the fraction of unbound water in the formulation. With the volume fraction of particles below 5% it is likely that some amount of osmotically active small molecules like e.g. lactose, have to be added to the formulation for it to be isoosmotic with blood (285 mOsm) which would be of benefit for the patient.

Other characteristics of dispersed forms of the composition of the invention are that the aqueous liquid phase is a) isoosmotic with the blood of the living organism to which the composition is to be administered, and b) devoid of diethylene glycol (DEG) and residues of unacceptable reactants, by-products and/or solvents from the manufacture of the core particles and/or from the coating process. The term “devoid of” means that the level of such contaminants in the composition is within limits as approved for this kind of composition by a regulatory official, such as FDA in the US or the corresponding authority in Japan or in one or more countries in Europe. For DEG this limit is likely to be below 0.2% of the composition which is the upper limit for DEG in compositions intended for human intake.

Certain variants of the composition are characterized in that the composition is adapted for administration to a living individual of the species discussed elsewhere in this specification. For animals this includes administration of compositions in dispersed form by injection, for instance to the circulation of the individual, e.g. by intravenous administration.

The composition is further characterized in line with the characteristics of the coat and the core particles.

With the metal oxide nanoparticles described herein it is possible to obtain a proton MR signal from an aqueous sample with a magnitude which is at least 50%, such as at least 100%, of the magnitude of the signal obtained for Gd³⁺-DTPA. Even higher MR signals can be envisaged, such as at least 150%, or at least 200%, or at least 300% or more of the corresponding Gd³⁺-DTPA signal. With respect to relaxation rates (1/T₁ and/or 1/T₂) it is possible to accomplish values that are at least 50%, such as at least 100% or at least 150% or at least 200% of the relaxation rate obtained for Gd³⁺-DTPA. The comparison is made between values obtained for the same Gd(III)-concentration and otherwise the same conditions as illustrated in the experimental part. Achievable values for the ratio r₂/rᵢ are ≦2 such as ≦1.5 or ≦1.3.

The innovative composition when in a form prepared for delivery to a customer is typically stable for more than 30 days, such as more than a year. Stability in this context primarily refers to decrease during the time period referred to a) in content of metal ion in the nanoparticles of the composition, and/or b) in ability of the coating to hinder release of metal ions. For (a) this means that the metal ion content of the nanoparticles at the end of the time period is ≧80%, preferably ≧90%, such as ≧95% or ≧99%, of the content at the start of the period, and for (b) that the half-life (t_{1/2}) of the nanoparticles after the time period referred to is ≧10 hours, such as ≧24 hours (one day) or ≧5 days or ≧7 days or ≧15 days, preferably ≧30 days or ≧a year. Measurement is as outlined in the experimental part.

### Coating Procedure

The manufacture of coated nanoparticles to be used in the method is the third main aspect of the invention. The manufacturing process comprises two main routes: a) the one-step route comprising using a silane reagent (coating precursor) directly introducing a desired organic group R on the core particle, and b) the multi-step route utilising a silane reagent (=coating precursor) comprising an organic group that needs to be modified in subsequent steps to obtain the desired group R of the final coating. Introduction according to the multi-step route includes step-wise introduction involving two or more steps in order to obtain a desired R group of the final coating. The manufacturing process may comprise a combination of the two routes, i.e. some of the R groups of the coating are introduced according to the one-step route and others according to the multi-step route. We have found that the one-step route is preferred, for instance at least one or as many as possible of the silane reagents (coating precursors) used should work according to the one-step rute, i.e. be according to (b2) below.

The coating procedure is a method for coating a population of core particles comprising metal oxide in their surface as discussed for the first aspect. The method comprises the steps of:
(i) providing said population of core particles,
(ii) contacting the core particles of the population with one, two, three or more different silane reagents (coating precursors), each of which exhibits,
   a) a reactive group that comprises the silicon atom of the reagent, such as an alkoxy silane group, and
   b) an organic group that
      b1) is different for the different silane reagents,
      b2) is to be a part of the final coating (is equal to an R group), or
      b3) is transformable to such a part (transformable to an R group), and
(iii) transforming the organic groups that
   a) derive from type (b3) silane reagents that have used in step (ii), and
   b) have become attached to said surface in step (ii)
to a part of said coating (=to an organic group R of said coat).

The reactive group is capable of attaching the organic group of the reagent to the core surface by an —O—Si—C— linkage where the oxygen atom becomes attached to a surface metal ion of a core particle and the carbon atom is part of the organic group of the silane reagent. The reactive group is typically of the same kind as the reactive groups defined by X₁, X₂, X₃ and X₄ in the reticulating agent discussed below. Step (ii) is taking place under conditions allowing this kind of attachment.

The reaction conditions are well known in the field and may include hydrolytic conditions in the presence of a trialkylamine and/or treating the reaction mixture with microwaves to locally heat the particles. Microwaves may be preferred for creating monolayers of silane groups directly attached to the surface of the core particles.

In a preferred variant the method comprises that
(a) at least one of the silane reagents is
   (i) according to (b2) and has a charged silane group, preferably a negatively charged silane group, or
   (ii) is according to (b3) and has a charged or non-charged silane group that is to be transformed to a charged silane group of the final coating, preferably to a negatively charged silane group, and
(b) at least one of the remaining silane reagents is according to (b2) and is non-charged or is according to (b3) and has a non-charged or charged silane group that is to be transformed to a non-charged group of the final coating.

The molar ratio between group (a) silane reagents and group (b) silane reagents is typically ≦20, preferably ≦1, and ≧0.1, such as ≧0.5. The reactions with the different silane reagents are preferably carried out under competition (simultaneously) for at least two of them (at least one of group (a) and at least one of group (b)).

At least one of the silane reagents used in the coating procedure may comprise an organic group that is branched. At least one of the branches of such a group may be charged, e.g. negatively charged.

The silane reagents used in the method have a silicon atom that preferably carries
a) three reactive groups each of which is capable of creating a siloxane linkage between silicon and a metal ion in the surface of a core particle, and
b) one silane group (monoalkyl silane).

The reactive groups may be selected amongst the same as the reactive groups in the tetra reactive silic acid derivatives discussed below. The preferences are the same.

In other for the invention less typical silane reagents there may be one or two reactive groups combined with three or two silane groups, respectively.

The silane group in at least one, preferably all, of the silane reagents used in step (ii) comprises a hydrophobic spacer group attached directly to the silicon atom and preferably a hydrophilic organic group attached to this spacer group. This spacer group and the hydrophilic organic group may be selected amongst the same structural elements as may be present in R, R′ and B of the coating. In preferred cases the spacer group and the hydrophilic organic group of a silane reagent are the same as B and R′, respectively, of the final coating.

Step (ii) for the different silane reagents may be carried out in sequence or simultaneously (=competitively). Simultaneous reactions include partial overlap, i.e. a portion of a subsequent silane reagent may be included in the reaction mixture before all of a previous silane reagent has been reacted, for instance adding a portion of a subsequent silane reagent together with a starting silane reagent.

A synthetic strategy to make a desired silane reagent is to add the corresponding silane, (XO)₃SiH to a suitable unsaturated compound such as methylacrylate (CH₂CHCOOCH₃) or the corresponding phosphorus or sulfur analog, in the presence of a catalyst such as Speier's catalyst (H₂PtCl₆6H₂O) or even better, PtO₂ as reported by Mioskowski et al. in Org. Lett. 2002, 4, 2117-2119. In some instances it may be advantageous to add the silicon containing moiety as the last step in the synthesis of the precursor but in other cases it may be more convenient to elaborate the structure further after the introduction of the silicon atom. Another option is to use a chlorosilane Cl₃SiH for the addition to the double bond, followed by substitution with an alcohol to yield the corresponding siloxane.

Typically, a monoalkyl silane reagent will give a coating that is cross-linked into a silica mesh, which covers the core surface but, because the surface of the core will not necessarily match the geometry of the siloxane needs completely, it will contain some defects. To stabilize the coating against degradation, a cross linking agent such as a derivative of silic acid that is tetra reactive with nucleophiles is introduced to stitch up as many of those defects as possible. The chemical reaction that links the coating precursors and a tetra reactive silic acid derivative into a network is the spontaneous condensation of silanol groups, SiOH, to dimers, SiOSi, with the concomitant loss of a water molecule.

The particles thus may be reacted in parallel (competitively) with or subsequent (consecutively) to step (ii) with a reticulating tetra reactive derivative of silic acid to create a stabilizing polysiloxane skeleton. Typical such reticulating reagents have the general formula Si(X₁,X₂,X₃,X₄) where each X when bound to silicon according to the formula represents a mixed anhydride function, an acid halide function, an ester function of silic acid or any other function of silic acid that can give the condensation reaction discussed in the previous paragraph. The reactive group comprising an X group bound to Si typically should be hydroxy-reactive to give an Si—O bond. In other words two, three or four of the Xs may be identical or different with each of them being selected amongst halogen, such as F, Cl, Br and I, alkoxy such as lower alkoxy, and acyloxy such as lower acyloxy, for instance with acyl being a fatty acid acyloxy (alkanoyl). Typical reagents of this kind are tetramethoxy orthosilicate (TMOS) and tetraethyloxy orthosilicate (TEOS).

### Visualization Techniques

The method of the invention for visualization of biological material is in particular beneficial for magnetic resonance imaging (MRI) but may also be applied to other imaging techniques utilizing contrast agents, e.g. computed tomography (CT), near-IR fluorescence imaging, positron emission spectroscopy (PET), microscopying etc. Advantageously, the particles of this invention may also be used as an X-ray contrast agent since there are paramagnetic metal oxides, such as gadolinium oxide, that has a higher molar X-ray extinction than iodine.

So far the greatest advantages of particles and compositions according to the invention have been accomplished when using them as positive contrast agents for the creation of T₁-weighted MR images.

The imaging step (ii) is preferably performed under conditions giving a spatial resolution that is within the intervals given above.

The biological material may be tissue materials, individual cells and other cell samples, organs etc deriving from dead or living material. The material may derive from organisms, such as plants, vertebrates and invertebrates, microorganisms etc. Typical vertebrates are mammals including human beings, avians, etc.

Step (i) is carried out according to principles that are well known in the field.

With respect to biological tissue material that is to be visualized when present in an intact animal (including human) or organ, step (i) typically means that the nanoparticles are injected in the form of a dispersion via a blood vessel (intra-arterially or intravenously). For intact animals also other routes may be useful, for instance intramuscularly, orally (with due care taken for protecting the nanoparticles when passing the stomach), intraperitoneally etc. The amount of nanoparticles administered depends on what to be visualized, for instance visualizing larger parts of a body or an organ typically requires larger amounts/doses than smaller parts. The animal is typically a vertebrate, such as a mammal, an avian, an amphibian, a fish etc including in particular humans and various kinds of domestic animals including pets.

### Populations of Core Particles

The term “core particle” encompasses a single core particle but also a core that may be construed of one or more smaller core particles (=clusters) hold together within the final nanoparticle. The terms “core” and “core particle” are used synonymously in this specification if not otherwise apparent from the context.

Individual core particles expose at least on their surfaces the metal oxide containing a transition metal ion as discussed above, with preference for the transition metal ion being a lanthanide (+III), such as gadolinium (III+). The lattice defined by a metal oxide of a particular transition metal ion may contain also other elements, such as other transition metal ions and/or anions replacing the particular transition metal ion and O²⁻, respectively, of the lattice. An admixture of gadolinium sulfide may improve the stability of the particles in an aqueous environment. Addition of other paramagnetic ions, e.g. iron and/or paramagnetic rare earth metal ions, and/or other lanthanides can be envisioned to improve the relaxation properties of the particles. Addition of minor amounts of silicate, vanadate, zirconate, or tungstate may affect the size distribution of the particles in an advantageous way.

Typically the molar content of a paramagnetic metal ion, such as a lanthanide (+III) like gadolinium (+III), in the core particles is ≧50%, such as ≧75% or ≧90% or ≧99% of the total content of the transition metal ions or paramagnetic metal ions in the core particles. See further our co-pending US provisional application cited above and the corresponding international application filed in parallel with the present specification. The purity with respect to additives that are non-paramagnetic may be at least 80% (w/w). The purity with respect to paramagnetic metal ions is at least 80% of the total content of transition metal ions.

Suitable transition metals are found among elements of Group 3b Sc, Y, La; Group 4b Ti, Zr, Hf; Group 5b V, Nb, Ta; Group 6b Cr, Mo, W; Group 7b Mn, Te, Re; Group 8 Fe, Ru, Os, Co, Rh, Ir, Ni, Pd, Pt; Group 1b Cu, Ag, Au; Group 2b Zn, Cd, Hg; and included in group 3b the lanthanides (La and elements 58-71) and the actinides (Ac, elements 89-103).

The term “lanthanides” (Ln) is in the context of the invention used synonymously with the term “rare earth metals” if not otherwise indicated. The term thus includes scandium (Sc), yttrium (Y) in addition to the true lanthanides that are considered to be elements 57-71.

The transition metal preferably should be capable of exhibiting paramagnetism and/or ferromagnetism when in oxide form. Examples of the former are in particular found amongst the lanthanides such as gadolinium. Examples of the latter are in particular found in group 8 (Fe, Co and Ni).

Uncoated core particles of the population used are smaller than the corresponding coated variants and typically have a mean geometric diameters (sizes) that is within the range of ≦20 nm or ≦10 nm or ≦8 nm with preference for ≦6 nm and, most ideally between 1 and 5 nm. The lower limits of these intervals are typically 0.5 nm or 1 nm. Measurement is as described in the experimental part.

Individual cores of the innovative composition should preferably contain one or more single crystalline domains (=crystallites) of the metal oxide discussed above. This does not exclude that an innovative population of nanoparticles may contain core particles that comprise amorphous structure together with core particles that comprise crystalline structure or both structures in the same core particle. Thus, in a typical composition to be used in the inventive method at least 10%, such as at least 25% or at least 50% or at least 75% of the core particles comprise crystalline structure. It can be envisaged that in preferred variants 100% or close to 100% of the cores of a population will exhibit crystalline structure, i.e. ≧75%, such as ≧80%, ≧90%.

The term “crystalline structure” includes crystalline-like structures where the crystal lattice is somewhat distorted from the ideal bulk structure due to the large fraction of surface atoms of small particles or where the particles contain typical crystal defects such as, point defects, line defects like screw and edge dislocations, or various planar defects.

The nanoparticles of a composition according to the invention may be porous or non-porous. Non-porosity in particular should apply to the metal oxide core of coated particles. A composition according to the invention may contain nanoparticles in which there are both porous and non-porous cores. Porosity refers to ability for water and/or other liquids to penetrate the core/coat.

The core particle as such can be synthesized according to known principles for metal oxide nanoparticles. See for instance Soderlind et al, J Colloid Interface Sci. 288 (20059 140-148; Feldmann, Adv. Funct. Mater. 13 (2003) 101-107; Bazzi et al, 102 (2003) 445-450; Bazzi et al, J Colloid Interface Sci. 273 (2004) 191-197; Louis et al, Chem. Mater. 17 (2005) 1673-1682; Pedersen et al, Surface Sci. 592 (2005) 124-140; WO 2005 0088314 (Perriat et al); WO 2006031190 (Uvdahl et al); and US 2004 0156784 (Haase et al); U.S. Pat. No. 6,638,494 (Pilgrimm et al).

In principle the synthetic route comprises the following steps: (i) mixing and dissolving a soluble salt, e.g. halide or nitrate, of the desired metal ion and an appropriate hydroxide, e.g. metal hydroxide such as LiOH and NaOH, in the appropriate solvent, (ii) formation of crystal nuclei (nucleation), and (iii) crystal growth. The solvent should be selected such that the desired metal oxide is insoluble compared to the starting salt and hydroxide compound. The various steps are carried out while heating the mixture to a temperature that typically differs between different steps. Step (iii) is typically starting while step (ii) is on-going. Size, size distribution and morphology (e.g. crystalline) of the particles will depend on temperature, concentrations, incubation times, additives etc. See the experimental part and the publications cited.

Promising preliminary results for the manufacture of core particles to be used in the invention have been accomplished by carrying out the three steps in a flow system comprising a first region for step (i), a second region for step (ii) and a third region for step (iii) and transporting the reaction mixture through the regions in the order given during the process. Individual regions may or may not have separate temperature control functions allowing independent heating of a region if necessary. The process can be run in a continuous mode. The use of miniaturised flow systems will facilitate still better control of variables that determine crystal growth, and are therefore important for obtaining particles having a desired size, size distribution and morphology (e.g. crystal structure). A miniaturised flow system comprises a microchannel in which the reactions are carried out. Microchannels typically have at least one cross-sectional dimension ≦1 mm.

Important advantages with using a flow system are that a) it can easily be designed to give high productivity, for instance by running the system in continuous mode and/or parallelizing two or more systems/microchannels, and b) it facilitates control of process variables and therefore makes it easier to obtain core particles of a predetermined quality.

Flow systems for preparing nanosized particles, e.g. of metal oxide, have previously been described. See Kawa et al., J. Nanoparticle Res. 5 (81-85) 2002; deMellow J. & A., Lab Chip 4 (2004) 11N-15N (review); Tanaka et al., Org. Lett. 9 (2007) 299-302.

The above-mentioned flow process for the manufacture of core particles for use in coated or uncoated form as contrast agents in the visualization of biological material constitutes the forth main aspect of the invention, with particular emphasis of various modes of the 1^{st} to 3^{rd} aspect.

### EXPERIMENTAL PART

### Gadolinium Oxide Particles

Surprisingly, it has turned out that it is advantageous for the reliability and reproducibility of the particle synthesis process, to avoid contact of the heated and basic solutions with air. This improves the color of the prepared particle solution from brown-yellow to colorless or, at most, a pale yellow. Also, the reproducibility of the process is enhanced and electron microscopy indicates that the crystals are more regular and show well developed crystal faces. The more well-defined surface of these crystals will make the coating more regular and hence better able to stabilize the crystals. We have also found it to be beneficial to substitute the sodium hydroxide in the process described in Bridot et al., J. Am. Chem. Soc. 2007, 129, 5076-5085, by lithium hydroxide. Unexpectedly, this further increases the fraction of crystals with well developed surfaces.

### Example 1

### Synthesis of DEG Coated Gd₂O₃ Particles Using Sodium Hydroxide

Diethylene glycol (DEG, 30 ml) and NaOH (0.3 g, 7.5 mmol), in a round bottom flask, equipped with a magnetic stiffing bar, are stirred under a stream of nitrogen for 30 minutes. The NaOH pellets are first crushed in a mortar and then the required amount is added. The mixture is stirred vigorously and the flask is immersed in a pre-heated oil bath for 30 minutes. The solids are then dissolved. The heating bath is then removed. In a separate flask, also with a nitrogen atmosphere and magnetic stiffing, GdCl₃.6H₂O (2.23 g, 6 mmol) is dissolved in DEG (30 ml) by heating to 140° C. under nitrogen for 1 hour. The temperature of the mixture is raised to 180° C. and the sodium hydroxide solution is added in one portion. The solution is vigorously stiffed, and kept at 180° C. for 4 hours and then allowed to cool under nitrogen.

### Example 2

### Synthesis of DEG Coated Gd₂O₃ Particles Using Lithium Hydroxide

Diethylene glycol (DEG, 30 ml) and LiOH (0.18 g, 7.5 mmol), in a round bottom flask, equipped with a magnetic stiffing bar, are stirred under a stream of nitrogen for 30 minutes. The mixture is stirred vigorously and the flask is immersed in a pre-heated oil bath for 30 minutes. The solids are then dissolved. The heating bath is then removed. In a separate flask, also with a nitrogen atmosphere and magnetic stiffing, GdCl₃.6H₂O (2.23 g, 6 mmol) is dissolved in DEG (30 ml) by heating to 140° C. under nitrogen for 1 hour. The temperature of the mixture is raised to 180° C. and the sodium hydroxide solution is added in one portion. The solution is vigorously stirred, and kept at 180° C. for 4 hours and then allowed to cool under nitrogen.

### Gadolinium-Terbium Oxide Nanoparticles

### Synthesis Procedure:

Terbium-doped gadolinium oxide nanoparticles are synthesized by applying a modified “polyol” method procedure developed by Bazzi et. al. (J. Colloid Interface Sci. 273 (2004) 191-197). For the 5% Tb-doped Gd₂O₃, 5.7 mmol of GdCl₃.6H₂O and 0.3 mmol of TbCl₃.6H₂O are dispersed in 30 mL of diethylene glycol (DEG), strongly stirred and heated in a silicon oil bath at 140-160° C. for 1 hour. Addition of 7.5 mmol of NaOH dissolved in 30 mL DEG follows. After complete dissolution of the compounds, the solution is refluxed at 180° C. for 4 hours under strong stirring, yielding a yellow-green transparent suspension. For the synthesis of 20% Tb-doped Gd₂O₃, the above procedure is also followed (but adding 1.1 mmol of TbCl₃.6H₂O) except for the addition of NaOH solution. To obtain a capped powdered form of the particles, the as-synthesized suspension is first centrifuged-filtered (0.22 μm) for 30 minutes at 40° C. until complete collection of the fluid. This step is done to remove any large size agglomeration of the particles. The filtered suspension is heated to 140-160° C. with stirring, and 1 mmol of NaOH with either 1.5 mmol of citric acid monohydrate (CA) or dinicotinic acid (NA) dissolved in a small amount of DEG is added. The solution is then refluxed at 180° C. for 30 minutes under strong stirring, yielding a whitish-green dispersion/precipitate. After washing and centrifuging in methanol for several times and then drying under vacuum, an off-white powder is collected.

### Characterization of Tb-Doped Nanoparticles

The rare-earth oxide synthesized Gd₂O₃ doped with terbium element has mostly circular shaped particles with an average size of 3-7 nm in diameter as revealed on high resolution transmission electron microscopy micrographs (TEM). The particles appear as a regular crystalline lattice, showing the (222) planes (d≈3.2 Å), superimposed on an amorphous background. The powders obtained after precipitation with either citric acid (CA) or dinicotinic (NA) acid reveal different morphologies under scanning electron microscopy (SEM). The CA-capped nanoparticles show porous sponge-like structures while the NA-capped nanoparticles appear like agglomerated spherical structures with open cavities.

The Tb-doping level and chemical composition of the nanoparticles are analyzed with X-ray photoelectron spectroscopy (XPS) and energy dispersive X-ray spectroscopy (EDX). The Tb to Gd atom ratios of 5% Tb- and 20% Tb-doped Gd₂O₃ are found to be 0.055±0.004 and 0.226±0.031, respectively. The results further show that Tb exists only as an ion serving as a dopant to the gadolinium oxide particle. Successful coating with DEG, CA and NA is verified by both XPS and IR analysis.

The photoluminescence (PL) spectra of the powder are consistent with earlier findings for similar nanoparticles with four emission peaks between 460 and 640 nm for excitation at 266 nm (Louis et al., Chem. Mater. 17 (2005) 1673-1682).

The nanoparticles can be coated covalently as said elsewhere in this specification, for instance with various bifunctional silanes as described for the iron containing nanoparticles studied in the subsequent patent example.

### Gadolinium-Iron Oxide Nanoparticles

### Synthesis Procedure:

The procedure is essentially as outlined in the publications cited above.

Reference particles (non-doped Gd₂O₃ nanoparticles): 2.71 g of Gd(NO₃)₃ or 2.2 g of GdCl₃ (6 mmol) is dissolved in 30 ml of DEG and heated under reflux and with magnetic stirring. Then 0.3 g of NaOH (7.5 mmol) in 30 ml of DEG is added, at 95° C. for Gd(NO₃)₃ and at 140° C. for GdCl₃. The reaction is then allowed to proceed at 140° C. for 1 h whereafter the temperature is raised to 180° C. for 4 h.

Fe doped Gd₂O₃ nanoparticles: Gadolinium nitrate Gd(NO₃)₃.6H₂O (1.9 mmol), Fe(NO₃)₃ (0.1 mmol), NaOH (2.5 mmol) and deionized water (six drops) are added to about 15 ml of diethylene glycol (DEG) (doping level (Fe/(Fe+Gd))=5%). The mixture is stirred and heated to 140° C. When the reactants are dissolved, the temperature is further increased to 180° C. and maintained constant for 4 hours. A precipitate is formed which is separated by centrifugation and washed several times with methanol.

Gd(NO₃)₃ can be replaced with GdCl₃ which is likely to result in smaller nanoparticles.

By increasing the Fe/(Fe+Gd) ratio in the reaction mixture to 10%, 20% and 50%, the doping level of the obtained nanoparticles is correspondingly increased.

Perovskite Gd₂O₃ nanoparticles (Fe doping level 50%): 1 mmol of GdCl₃.6H₂O and 1 mmol of FeCl₃.6H₂O are added to 10 ml of DEG and heated. When the temperature reaches 180° C., 6 mmol of KOH dissolved in 10 ml of DEG is added. The temperature is further raised to 210° C. and kept at this temperature for 4 h. A dark brown precipitate is formed, separated off by centrifugation and washed twice with methanol. A certain amount of the sample is calcined at 800° C. in air for 3 h. The supernatant from the centrifuging is heated at 500° C. for 4 h, and the brown powder obtained is washed with deionised water.

X-ray diffractograms (XRD) show peaks attributable to the presence of perovskite, garnet and normal Gd₂O₃ crystal structure in varying amounts in particle material obtained from equimolar amounts of GdCl₃ and FeCl₃. The XRD measurements are performed on a Philips APD powder diffractometer, using CuK_{α} radiation (λ=1.5418 Å, 40 kV, 40 mA) and a step-size of 0.025° in 2θ with 4 s/step.

### Working Up of Nanoparticles

Synthesized nanoparticles are centrifuged (Hermle Z513K) using Vivaspin concentrator membrane (polyethersulfone or PES, Vivascience Sartorius, Hannover) for 30 min. Filters with pore size 0.2 μm, 100 000 molecular weight cut off (MWCO) and 50000 molecular weight cut off (MWCO) are used. The speed is set to 1750 rpm and the temperature is set to 40° C. A syringe driven filter with pore size 0.22 μm (Millex® GV Filter Unit 0.22 μm. Durapore® PVDF membrane, Millipore, Corrigtwohill) is also tested. The results are evaluated using dynamic light scattering (DLS).

Dialysis is performed both to remove excess DEG and in later steps unreacted molecules used for functionalization (e.g. silanes). To remove DEG, the suspension is dialyzed against Milli-Q water with a 1000 MWCO membrane (SpectraPor 6, flat width 18 mm, SpectrumLabs, Rancho Dominguez Calif.) on a magnetic stirrer. The water is replaced at least three times the first day and then two times every following day. The ratio of nanoparticle suspension to water is ideally 1:1000. To evaluate the effect of dialysis time on agglomeration, a nanoparticles suspension filtered with Vivaspin 0.2 μm is dialyzed for 48, 72 and 96 h and the result is evaluated using DLS. To remove unreacted species after functionalization steps, both 1000 MWCO and 10 000 MWCO filters are used. Membranes 10 000 MWCO with a flat width of 12 mm and 18 mm are used. The former gives a quicker dialysis but the latter is easier to use and less expensive. Dialyzed suspensions are stored at 4° C.

Size fractionation: The nanoparticles of a batch can be fractionated into size fractions by using Vivaspin 20 ultrafiltration spin columns in a Rotina 35R Centrifuge (Hettich Centrifugen) and filters of decreasing MWCO by filtrating nanoparticles in the filtrate from a filter of higher MWCO through a filter of lower MWCO. The filters of 100000 MWCO, 50000 MWCO, 30000 MWCO and 10000 MWCO which correspond to cut-off sizes 13.3 nm, 6.67 nm, 4 nm, and 1.33 nm when used consecutively in the given order will thus give four defined size fractions, i.e. nanoparticles collected on each filter plus the nanoparticles in the filtrate passing through the 10000 MWCO. The nanoparticles collected on top of the 100000 MWCO filter are discarded since they contain various types of aggregates of undefined sizes and composition.

### Measurement of Particle Sizes

This is carried out by dynamic light scattering (DLS) and transmission electron microscopy (TEM). DLS: The particle size of a colloidal suspension of the above-mentioned perovskite (not heated to 800° C.) material is measured in AV/DLS-5000 system (Lange). The optimal counting rate is about 250 mHz, and normalized intensity correlation function curves are carefully fitted with an exponential algorithm of the second order (200 grid points). The hydrodymic radius for particles of the suspension is found to be 4.8±0.3 and 5.7±1.0 nm. TEM: These studies are carried out with a Philips CM20 ST electron microscope, operated at 200 kV, and a FEI Tecnai G2 electron microscope (200 kV). Samples for TEM analysis are prepared by dissolving in methanol as-synthesized, non-dialyzed products. The dispersion is dried on amorphous carbon-covered copper grids. By the use of TEM images taken at about 500000× magnification size distribution histograms are built from which an average size can be estimated. An average size of 3.5 to 4.0 nm is estimated (crystal core) for the perovskite material.

### Functionalization of Nanoparticles:

a) Silanization of the nanoparticles by the use of a hetero bifunctional silane with a subsequent further functionalization, e.g. PEG-ylation (two-step PEG-ylation procedure). Filtered nanoparticles are sonicated for 15 minutes, in order to break agglomerates. 1 ml of the nanoparticles in a water suspension (typically dialysed in a previous step) is then placed in an eppendorf tube and 50 μl of the bifunctional silane, e.g. 3-aminopropyl triethoxy silane, is added followed by vortexing and 1 h of sonication. During the reaction the silane function binds to the surface of the nanoparticles leaving the other function, e.g. an amino function, free for the subsequent functionalization step, e.g. introduction of hydrophilic polymers such as polyethylene glycol (PEG-ylation). If needed the silane is added together with a solvent with due care taken for favouring reaction between silane and nanoparticles compared to polymerisation of the silane. 10 μL of Milli-Q is then added whereafter the suspension is sonicated for 1 h and placed on a mixer table overnight to give a total reaction time of 20 h. Purification of the silane-coated particles is performed by dialysis against Milli-Q for 48 h with a 1000 MWCO membrane. The same procedure is also performed with 0.5 and 10 μL of the silane. This functionalization is done with 3-aminopropyl triethoxy silane (APTES).
b) Silanization by the use of a bifunctional PEG derivative (one-step PEG-ylation procedure). 3-mercaptopropyl triethoxysilane (MPTES) and a hetero bifunctional Mal-PEG-NHS derivative (Mal=N-maleidyl linked via a spacer (—(CH₂)ₙCO—) to the oxygen in one terminal of PEG and N-succinimidyl linked via a spacer (—CH₂)_{n′}COO—) to the oxygen in the other terminal of PEG (n and n′=an integer >0) are in a prestep reacted with each other under conditions permitting the mercapto group to form a thioether bond with the C—C double bond in Mal. 15 mg of Mal-PEG-NHS (3 μmol) is dissolved in 300 μL, of ethanol using sonication since heat is required to achieve dissolution. Then 0.5 μL, of MPTES is added and the reaction is allowed to proceed for 1 h in an ultrasonic bath. Next, 1 ml of Gd₂O₃-DEG nanoparticle suspension, filtered and dialyzed for 72 h, is added followed by vortexing and sonication for 2 h. The tube is then placed on a mixer table overnight to give a total incubation time of at least 20 h. To remove excess of Mal-PEG-NHS and MPTES, dialysis is performed against Milli-Q for 48 h using a 10 000 MWCO membrane. The same procedure is done using 5 mg of Mal-PEG-NHS with 0.05 μL, MPTES and 10 mg of Mal-PEG-NHS with 0.1 μL, MPTES. The NHS group of the thus NHS functionalized nanoparticles can then be further functionalised with targeting groups, labels such as fluorophors and the like, etc exhibiting an amino group.
c) Silanization by the use of PEG silanes, such as PEG-triethoxy silane (one-step PEG-ylation procedure). This kind of silanes (Mw_{PEG}=4000 and 5000 daltons) is reacted as outlined above for other silanes, for instance with the PEG moiety in mono methoxylated form.

### Magnetic Properties and Stability of Nanoparticles

Measurement of stability/dissolution of nanoparticles: The desired nanoparticles synthesized as described above and dispersed in MilliQ water are prepared for seven days of dialysis (1000 MWCO dialysis membrane). The concentration/content of Gd(III) in the dispersion as a function of dialysis time is determined at three different occasions i.e. before dialysis, after five days and after seven days. The dialysis is performed at room temperature. The Gd content in the nanoparticle suspension is analyzed by Inductively Coupled Plasma Mass Spectrometry (ICP-MS), Analytica.

MRI measurements: See Engström et al., Magn Reson Mater Phy, 19 (2006) 180-186.

### Results of Comparative Studies of Different Nanoparticles (Relaxation Rates and Stability):

The results indicate that paramagnetic nanoparticles suitable for magnetic resonance imaging can be synthesized with predetermined and/or improved properties, e.g. with predetermined and/or improved relaxation rates (1/T₁ and 1/T₂), relaxivities (r₁ and r₂) and stability/lifetimes. This is illustrated by the finding that a) PEG silane functionalized Gd₂O₃ nanoparticles have a high 1/T₁ and 1/T₂ (T₁ (1 mM)=0.012 ms⁻¹) and a fast dissolution rate (short lifetime) (t_{1/2}=4 days), b) PEG silane functionalised 5% Fe doped Gd₂O₃ nanoparticles have a high 1/T₁ and 1/T₂ (1/T₁ (1 mM)=0.012 ms⁻¹) and a considerably slower dissolution rate (longer lifetime (t_{1/2}=10 days), and c) DEG coated non-doped Gd₂O₃ nanoparticles have 1/T₁ (1 mM)=0.012 ms⁻¹ and t_{1/2}=14 days. Commercially available and clinically used Gd³⁺-DPTA has under the same conditions lower values for 1/T₁ and 1/T₂ (e.g. 1/T₁=0.005 ms⁻¹). Variations in relaxivities (r₁ and r₂) and in the relaxivity ratio (r₂/r₁) are illustrated by:

| | r₁ | r₂ | |
|---|---|---|---|
| Nanoparticles | mM⁻¹s⁻¹ | mM⁻¹s⁻¹ | r₂/r₁ |
| Gd³⁺-DTPA | 4.1 | 4.7 | 1.1 |
| Gd³⁺ (GDCl₃) | 10.5 | 12.4 | 1.2 |
| Gd₂O₃ PEG-silane dialyzed 120 h | 9.4 | 13.4 | 1.4 |
| GdFeO₃ dialyzed 4 h | 5.1 | 6.1 | 1.2 |
| GdFeO₃ 800° C. dialyzed 4 h | 6.1 | 10.6 | 1.6 |
| GdFeO₃ dialyzed 120 h | 11.9 | 15.2 | 1.3 |
| Gd₂O₃ 5% Fe* dialyzed 16 h | 5.1 | 6.1 | 1.2 |
| Gd₂O₃ 5% Fe* PEG-silane, dialyzed | 6.1 | 10.0 | 1.6 |
| 120 h | | | |

*The synthesis is the same as for GdFeO₃ (perovskite) except that the relative amount of Fe3+ is lowered to 5%.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method for visualizing biological material, preferably by MRI, comprising the steps of:
(i) bringing a population of coated nanoparticles into contact with said biological material, each of which nanoparticles comprises a) a metal oxide of a transition metal, said metal oxide preferably being paramagnetic and preferably comprising a lanthanide (+III) such as gadolinium (+III), and b) a coat covering the surface of the core particle, and
(ii) recording the image;
wherein the coat is hydrophilic and comprises a silane layer which is located next to the surface of the core particle and comprises one, two or more different silane groups each of which comprises an organic group R and a silane-siloxane linkage —O—Si—C— where
a) the organic group R comprises a hydrophilic organic group R′ and a hydrophobic spacer B,
b) O is an oxygen atom directly binding to a surface metal ion of the metal oxide, and
c) C is a carbon atom and is also part of the hydrophobic spacer B.

2. The method of claim 1, wherein the core particles have a mean geometric diameter ≦20 nm, preferably ≦10 nm, such as ≦8 nm, and ≧0.5 nm, such as ≧1 nm.

3. The method of claim 1, wherein the nanoparticles (coated core particles) have a mean hydrodynamic diameter ≦20 nm, preferably ≦10 nm, such as ≦6 nm, and ≧0.5 nm, such as ≧1 nm.

4. The method of claim 1, wherein the coat has a thickness which is ≦10 nm, such as ≦5 nm or ≦1 nm or ≦0.7 nm with a typical lower limit of 0.1 nm or 0.5 nm.

5. The method of claim 1, wherein the coat has a thickness in the range of a monolayer.

6. The method of claim 1, wherein the molar ratio between silicon in the coat and metal ions in the core particles is ≧50%, such as ≧80% or ≧90% and typically ≦1000%, such as ≦250% or ≦150%, of the maximum value for the molar ratio between silicon bound via oxygen directly to a metal ion in the surfaces of the core particles and metal ions in the core particles.

7. The method of claim 1, wherein the molar ratio between silicon bound via oxygen directly to a metal ion in the surface of the core particle and metal ions in the core particle is ≧50%, such as ≧80% or ≧90% and ≦100% of the maximum value for this ratio.

8. The method of claim 1, wherein the molar ratio between silicon in the coat and carbon bound directly to silicon (silane carbon) that for instance is directly attached to surface metal ions of the core particle via oxygen, is ≧1 and typically ≦5 such as ≦2.5 or ≦1.5, with preference for ≦1.25 or ≦1.1

9. The method of claim 1, wherein the hydrophobic spacer B complies with
—(CₙH₂ₙ₋₂ₐ)— (Formula I)
where one, two or two more hydrogens possibly is/are substituted with a lower alkyl or a lower alkylene group, respectively, with n being an integer 1-15, preferably an integer 1, 2, 3, 4 or 5, and a is an integer 0, 1, 2, 3, etc with a ≦n.

10. The method of claim 1, wherein said hydrophilic organic group R′ in said one, two or more silane groups comprises a carbon chain which at one, two or more positions
a) is interrupted by an at least bivalent functional group containing a heteroatom selected amongst O, N, and S, and/or
b) comprises a carbon that is
(i) substituted with hydroxyl or lower alkoxy possibly substituted with hydroxy or amino, possibly substituted with lower alkyl possibly substituted with hydroxy,
(ii) a branch point of the carbon chain with a branch group that comprises structural elements that are selected amongst the same structural elements as may be present in the hydrophilic organic group.

11. The method of claim 1, wherein said hydrophilic organic group R′ in at least one of said one, two or more silane groups comprises a charged group, preferably in an amount and combination giving the nanoparticles an absolute zeta potential ≧20 mV, such as ≧30 mV.

12. The method of claim 1, wherein the hydrophilic organic group R′ in at least one of said one, two or more silane groups is selected amongst groups complying with the formula:
-(ACH₂CH₂)ₚ(OCH₂CH₂)ₘA′ₒ(CH₂)_{n′}X Formula II)
where
a) n′ is an integer 0-15, preferably 1-5,
b) m is an integer 0-10, preferably 2-5,
c) o and p are equal or different integers 0 or 1, with the proviso that one of them preferably is 0 when m is 0;
d) A and A′ are heteroatom-containing bifunctional groups with said heteroatom being selected amongst oxygen, nitrogen and sulphur and with preference for the bifunctional group being ether, thioether or amino, and
e) X is selected amongst carboxylate alkylester, phosphonate alkyl ester (mono or dialkyl), sulphonate alkyl ester, N-alkyl amide (mono or dialkyl), N-alkyl phosphonic acid amide (mono- or dialkyl), N-alkyl sulphonamide, alkyl ether and the corresponding hydrolysed forms.

13. The method according to claim 1, wherein the hydrophilic organic group R′ in at least one of said one, two or more silane groups is branched, for instance with one or more of the hydrogens in formula II independently from each other being replaced with a group complying with formula II at one or more positions (one, two or more branch points).

14. The method of claim 1, wherein the coated nanoparticles and/or the core particles are monodisperse.

15. A method of coating a population of core particles comprising paramagnetic metal oxide in their surface, which method comprises the steps of:
(i) providing said population of core particles,
(ii) contacting the core particles with one, two, three or more different silane reagents, each of which exhibits,
a) a reactive group comprising the silicon of the silane reagent, and
b) an organic group that
b1) is different for the different silane reagents,
b2) is to be a part of the final coat (is equal to an R group), or
b3) is transformable to such a part (transformable to an R group),
said contacting taking place under conditions allowing direct attachment of said organic group of each of said silane reagents to the surfaces of said core particles by —O—Si—C— linkages, and
(iii) transforming said organic groups if being according to (b3) to a part of said coat (=to an R group of said coat).

16. The method according to claim 15, wherein step (ii) for the different silane reagents is carried out simultaneously (=competitively).

17. The method according to claim 15, wherein the particles are reacted with a reticulating reactive silicate, such as tetraalkyl orthosilicate either simultaneously (competitively) with or subsequently to step (ii).

18. The method according to claim 15, wherein at least one of said silane reagents is according to b2.

19. The method according to claim 15, wherein at least one of said silane reagents comprises a hydrophobic spacer group attached directly to its silicon atom.

20. The method according to claim 15, wherein at least one of said silane reagents comprises an organic group comprising a hydrophobic spacer group attached directly to its silicon atom and a hydrophilic group attached to said spacer group.

21. The method according to claim 15, wherein said organic group, said spacer group and said hydrophilic group to the extent they are present in one or more of said silane reagents are as defined for R, R′ and B in any of claims 1 and 9-13.

22. The method according to claim 15, wherein
(a) at least one of the silane reagents is according to (b2) and comprises a charged group, preferably a negatively charged group, and
(b) at least one of the remaining silane reagents to the extent such reagents are used is according to (b2) and non-charged.

23. The method according to claim 22, wherein the molar ratio between group (a) silane reagents and group (b) silane reagents is ≦20, preferably ≦1, and ≧0.5, such as ≧0.1, and preferably performing the reaction of at least two said silane reagents with the particles under competition (at least one of group (a) and at least one of group (b)).

24. The method according to claim 15, wherein the particles are reacted with a reticulating reactive silicate, such as tetraalkyl orthosilicate either simultaneously (competitively) with or subsequently to step (ii) and wherein the molar ratio between the reticulating reactive silicate and the sum of the silane reagents is 0-0.5.

25. The method according to claim 15, wherein at least one of the silane reagents comprises an organic group that is branched.

26. A composition intended for visualization of biological material, typically for use as a contrast agent in in-vivo imaging, such as MRI, X-ray, PET, CT and fluorescence imaging, with preference for MRI and X-ray, wherein the composition comprises a population of nanoparticles as defined in claim 1.

27. The composition of claim 26, wherein the nanoparticles are dispersed in a physiologically acceptable aqueous liquid phase with a concentration of the transition metal ion of the metal oxide being ≧500 mM, with preference for ≧1M, said metal ion typically being a lanthanide (+III) with preference for gadolinium (+III).

28. The composition of claim 27, wherein the liquid phase is isoosmotic with blood of the organism to which the composition is to be administered.

29. The composition of claim 26, wherein it is devoid of solvent residues originating from the manufacture of the core particles.

30. The composition of claim 26, wherein it is devoid of diethylene glycol (DEG).

31. The composition of claim 26, wherein its viscosity at a concentration of 0.5 M of the metal ion of the metal oxide is ≦50 mPas, preferably ≦25 mPas or ≦15 mPas.

32. The composition of claim 26, wherein the core particles have been manufactured by a continuous flow process.

33. The composition of claim 26, wherein the core particles have been produced under nitrogen atmosphere.

34. The composition of claim 26, wherein the nanoparticles are stable in aqueous solution for ≧one month, such as ≧one year.

35. The composition of claim 26, wherein ≧50%, such as ≧80% or ≧90% of the nanoparticles are excreted within 48 hours from the body of the living organism to which they are to be administered.
